(19) United States
(12) Patent Application Publication
(10) Pub. No.: US 2026/0263511 A1
(43) Pub. Date: Sep. 10, 2026

(54) COMPOSITION FOR TREATING ATOPIC DERMATITIS

(71) Applicant: HYPHENS PHARMA PTE. LTD., Singapore (SG)

(72) Inventor: Sue Phay NG, Singapore (SG)

(73) Assignee: HYPHENS PHARMA PTE. LTD., Singapore (SG)

(21) Appl. No.: 18/843,068

(22) PCT Filed: Feb. 24, 2023
(86) PCT No.: PCT/EP2023/054686

§ 371 Date: Aug. 30, 2024

(30) Foreign Application Priority Data
Mar. 3, 2022 (GB) 2202970.6

(51) Int. Cl. A61K 33/30; A61K 9/00; A61K 31/366; A61K 31/7032; A61K 47/02; A61K 47/06; A61K 47/10; A61K 47/14; A61K 47/18; A61K 47/26; A61K 47/28; A61P 17/00

(52) U.S. Cl. CPC A61K 33/30; A61K 9/0014; A61K 31/366; A61K 31/7032; A61K 47/02; A61K 47/06; A61K 47/10; A61K 47/14; A61K 47/18; A61K 47/26; A61K 47/28; A61P 17/00

(57) ABSTRACT

A composition for use in a method of treating, or methods of treating, atopic dermatitis, wherein the composition is topically applied on the skin at least 2 times a day, wherein the composition has a pH in the range of 2.7 to 5.0 and includes a polyhydroxy acid and a zinc salt of its conjugate base, wherein the polyhydroxy acid is selected from lactobionic acid, gluconolactone, or a combination of lactobionic acid and gluconolactone, a partition coefficient enhancer selected from propylene glycol, butylene glycol, pentylene glycol, hexylene glycol, 1,5 pentane diol or a combination thereof, a diffusion coefficient enhancer which is selected from a C12 to C14 straight chain fatty acid or a C14 straight chain primary alcohol. The method of treating is of particular use for the treatment of moderate atopic dermatitis and/or for topically treating the skin between 10 and up to 28 days.

### BACKGROUND

Atopic dermatitis, otherwise known as eczema, is the most common inflammatory skin disease condition worldwide. The condition is characterized by one or more of dry, cracked or swollen skin, itching, red patches, and blisters. Atopic dermatitis typically begins in infancy, but affects up to 20% of children, although other ages can be affected.

However, present treatment for most atopic dermatitis, particularly moderate and severe atopic dermatitis, relies on the application of steroid creams. However, these treatments generally requires the recommendation of a doctor or medical practitioner and in some instances not recommended for very young children. The application of steroid creams is often associated with side-effects such as burning or stinging, while prolonged use can lead to stretch marks or skin thinning which is undesirable as atopic dermatitis sufferers already have compromised skin barrier. Other common topical compositions for atopic dermatitis include medicated creams. These creams are anti-inflammatory agent and immunomodulators, for example comprising calcineurin inhibitors, or antiallergenic agents such as antihistamines. However, medicated creams can similarly cause skin irritation, lead to skin thinning, dilation of blood vessels, increased skin fragility and increased risk of infection. They are generally not recommended for prolonged use.

Some moisturizing or emollient creams are proposed for use in the treatment and prevention of mild atopic dermatitis, to reduce the likelihood of flares in eczema-prone skin. However, some of these treatments have been shown to have little effect, require frequent application and/or require or recommend a prolonged treatment of at least 4 weeks for any meaningful improvement to be seen. For the treatment of moderate or severe atopic dermatitis, moisturizing or emollient creams are only to be used as part of a combination therapy with different pharmaceuticals, such as steroids (See Hebert et al, American Journal of Clinical Dermatology, (2020), 21:641-655).

### SUMMARY OF INVENTION

In accordance with a first aspect of the present invention, there is provided a composition for use in a method of treating moderate atopic dermatitis (and/or atopic dermatitis with a local SCORAD score of at least 6), wherein the composition is topically applied on the skin of a subject at least 2 times a day, wherein the composition has a pH in the range of 2.7 to 5.0 and comprises
a polyhydroxy acid and a zinc salt of its conjugate base, wherein the polyhydroxy acid is selected from lactobionic acid, gluconolactone, or a combination of lactobionic acid and gluconolactone,
a partition coefficient enhancer selected from propylene glycol, butylene glycol, pentylene glycol, hexylene glycol, 1,5 pentane diol or a combination thereof, and a diffusion coefficient enhancer which is selected from a C12 to C14 straight chain fatty acid or a C14 straight chain primary alcohol.

In accordance with a second aspect of the present invention, there is provided a method of treating moderate atopic dermatitis (and/or atopic dermatitis with a local SCORAD score of at least 6), wherein the method comprises topically applying the composition on the skin of a subject at least 2 times a day for between 10 and up to 28 days, wherein the composition has a pH in the range of 2.7 to 5.0 and comprises
a polyhydroxy acid and a zinc salt of its conjugate base, wherein the polyhydroxy acid is selected from lactobionic acid, gluconolactone, or a combination of lactobionic acid and gluconolactone,
a partition coefficient enhancer selected from propylene glycol, butylene glycol, pentylene glycol, hexylene glycol, 1,5 pentane diol or a combination thereof,
a diffusion coefficient enhancer which is selected from a C12 to C14 straight chain fatty acid or a C14 straight chain primary alcohol.

In accordance with the first or second aspect, in some embodiments, the method of treatment may be for between about 5 and up to 28 days, or between 10 and up to 28 days, or between about 10 and 25 days, or between 10 and 20 days, or between 11 and 18 days, or between 12 and 16 days. In some embodiments, the method of treatment may be for less than 28 days, or less than 25 days, or less than 22 days, or less than 20 days, or less than 18 days, or less than 16 days, or about 14 days.

In accordance with a third aspect of the present invention, there is provided a composition for use in a method of treating atopic dermatitis, wherein the composition is topically applied on the skin of a subject for between 10 and up to 28 days at least 2 times a day, wherein the composition has a pH in the range of 2.7 to 5.0 and comprises
a polyhydroxy acid and a zinc salt of its conjugate base, wherein the polyhydroxy acid is selected from lactobionic acid, gluconolactone, a combination of lactobionic acid and gluconolactone,
a partition coefficient enhancer selected from propylene glycol, butylene glycol, pentylene glycol, hexylene glycol, 1,5 pentane diol or a combination thereof,
a diffusion coefficient enhancer which is selected from a C12 to C14 straight chain fatty acid or a C14 straight chain primary alcohol.

In accordance with a fourth aspect of the present invention, there is provided a method of treating atopic dermatitis, wherein the method comprises topically applying the composition on the skin for between 10 and up to 28 days at least 2 times a day, wherein the composition has a pH in the range of 2.7 to 5.0 and comprises
a polyhydroxy acid and a zinc salt of its conjugate base, wherein the polyhydroxy acid is selected from lactobionic acid, gluconolactone, a combination of lactobionic acid and gluconolactone,
a partition coefficient enhancer selected from propylene glycol, butylene glycol, pentylene glycol, hexylene glycol, 1,5 pentane diol or a combination thereof,
a diffusion coefficient enhancer which is selected from a C12 to C14 straight chain fatty acid or a C14 straight chain primary alcohol.

In accordance with the third or fourth aspect, in some embodiments, the atopic dermatitis can be mild, mild-moderate, moderate, moderate-severe or severe atopic dermatitis. In some embodiments, the atopic dermatitis is moderate atopic dermatitis. In accordance with the third or fourth aspect, in some embodiments, the method of treatment may be for between about 10 and 25 days, or between 10 and 20 days, or between 11 and 18 days, or between 12 and 16 days. In some embodiments, the method of treatment may be for less than 28 days, or less than 25 days, or less than 22 days, or less than 20 days, or less than 18 days, or less than 16 days, or about 14 days. The atopic dermatitis may be characterized with a local SCORAD score of at least 6, or between 6 and 12. The atopic dermatitis may be moderate, moderate-severe, or severe atopic dermatitis

Also disclosed herein, is a composition for use in a method of treating, or a method of treating, atopic dermatitis characterized with a local SCORAD score of at least 6 at the start of treatment, wherein the composition is topically applied on the skin of a subject at least 2 times a day, wherein the composition has a pH in the range of 2.7 to 5.0 and comprises
a polyhydroxy acid and a zinc salt of its conjugate base, wherein the polyhydroxy acid is selected from lactobionic acid, gluconolactone, or a combination of lactobionic acid and gluconolactone,
a partition coefficient enhancer selected from propylene glycol, butylene glycol, pentylene glycol, hexylene glycol, 1,5 pentane diol or a combination thereof, and
a diffusion coefficient enhancer which is selected from a C12 to C14 straight chain fatty acid or a C14 straight chain primary alcohol.

Also disclosed herein, is a composition for use in the manufacture of a medicament for treating atopic dermatitis (e.g., moderate atopic dermatitis or atopic dermatitis characterized by a local SCORAD score of at least 6, for example, between 6 and 12), wherein the composition is topically applied on the skin of a subject at least 2 times a day, wherein the composition has a pH in the range of 2.7 to 5.0 and comprises
a polyhydroxy acid and a zinc salt of its conjugate base, wherein the polyhydroxy acid is selected from lactobionic acid, gluconolactone, or a combination of lactobionic acid and gluconolactone.
a partition coefficient enhancer selected from propylene glycol, butylene glycol, pentylene glycol, hexylene glycol, 1,5 pentane diol or a combination thereof, and
a diffusion coefficient enhancer which is selected from a C12 to C14 straight chain fatty acid or a C14 straight chain primary alcohol.

Also disclosed herein is a composition comprising
4-6% w/w polyhydroxy acid and zinc salt of its conjugate base, wherein the polyhydroxy acid is lactobionic acid
15-20% w/w partition coefficient enhancer which is butylene glycol
1-2% w/w diffusion coefficient enhancer, preferably tetradecyl alcohol, and
10-20% w/w emollient, preferably hydrogenated polydecene
4.5-12% w/w surfactant, preferably wherein the surfactant comprises one or more of methyl glucose sesquistearate, PEG-20 methyl glucose sesquistearate, glyceryl stearate or a combination thereof,
40-60% w/w water, and
up to 3% w/w humectant, preferably glycerol, and
a co-surfactant, preferably in an amount up to 6% w/w of the composition
a ceramide, in an amount from 0.5% to 1.5% w/w of the composition cholesterol, preferably in an amount up to 0.5% w/w of the composition
a fatty acid, preferably in an amount up to 0.5% w/w of the composition a PEG/HDI copolymer, preferably a PEG-240/HDI copolymer bis-decyltetradeceth-20 ether, preferably in an amount up to 1% w/w of the composition
a acrylates/C10-C30 alkyl acrylate copolymer, preferably in an amount up to 0.5% w/w of the composition, and
optionally a preservative, preferably in an amount up to 1% w/w of the composition
wherein the pH of the composition is between 3.0 and 3.5.

### Advantages of the Present Invention

The present invention provides a composition for use in an improved method of treatment that provides patients with a statistically significant improvement in atopic dermatitis, particularly moderate atopic dermatitis and/or atopic dermatitis with a local SCORAD score of at least 6. The composition can be used with patients with moderate atopic dermatitis showing a meaningful improvement in symptoms in a very short timescale, thereby indicating that the cream shows better results compared to other moisturising creams on the market. As shown in the Examples of the application as filed (see Example 1), topical application of the composition in clinical trials resulted in a local SCORAD score reduction from an average of 8 (indicative of moderate eczema) to an average of 4 (indicative of mild eczema) in as little as 2 weeks. As far as the inventors are aware, this is the first time that topical composition has been able to provide such good results in the absence of a steroid, particularly in such a short timescale. Other moisturising creams typically require a treatment duration of at least 4 weeks to see meaningful improvement if a steroid is not used.

The composition for use or the method of treatment as described herein leads to one or more of a more marked decrease in dryness, erythema/redness, edema and papules, weeping and crusts, and lichenification after 2 weeks of treatment as compared to the start of treatment/baseline. In most patients, the composition for use or the method treatment leads to a marked decrease in multiple symptoms after 2 weeks of treatment as compared to the start of treatment/baseline.

While similar compositions have previously been reported, there has previously no indication that said compositions could be used to treat patients with moderate atopic dermatitis, let alone in such a short timescale. There was no such indication that a similar composition could be used for successful treatment of moderate atopic dermatitis, particularly with significantly improved results within such a short timescale, especially in the absence of other pharmaceutical actives such as hydrocortisone. The statistically significant improvement in local SCORAD score for patients suffering from moderate atopic dermatitis was not expected, particularly in the absence of other pharmaceutical actives such as steroids, or hydrocortisone/hydrocorticosteroid treatment. Thus, the present invention provides use of the composition for a new patient group (i.e., patients suffering from moderate atopic dermatitis, or patients with a SCORAD score of at least 6, preferably between 6 and 12) not previously anticipated or expected. In certain embodiments or aspects, the present invention also provides for a new dosage regime where the composition is applied for a specific time period, that is, up to 4 weeks, and preferably in a shorter timescale, such as about 2 weeks. In certain embodiments or aspects, the composition for use and methods of treatment disclosed herein can provide a much milder and less irritating treatment as compared to other treatments used in the prior art such as methods of treatment that include other pharmaceutical actives or steroid treatment.

The composition is able to significantly lower skin pH and allow the skin barrier to improve.

The composition for use is also improved as compared to the previously disclosed compositions. The advantages of the composition for use in the treatment of atopic dermatitis can be attributed to the specific combination of ingredients. Furthermore, different to previously disclosed compositions, the use of butylene glycol has less restrictions in young infants and found to be less irritating as compared to other partition coefficient enhancers (e.g., propylene glycol). The use of an improved formulation showed improvements in both the stability and texture of the composition.

### BRIEF DESCRIPTION OF FIGURES

FIG. 1 shows the allocation of sites for treatment used in the study detailed in Example 1 for arms, although the sites may equally be applied to the legs. A=untreated lesion skin, B=treated lesion skin.

FIG. 2 shows a Local SCORAD bar chat with mean values and 95% confidence intervals of sum scores (n=29) between baseline (day 1) and D15 (day 15), when applied to A=untreated lesion skin or B-treated lesion skin.

FIG. 3 shows a bar chart with mean values and 95% of confidence intervals of differences in Local SCORAD to baseline (day 1) at D15 (day 15), when applied to A=untreated lesion skin or B-treated lesion skin.

FIG. 4 shows a bar chart with mean values of differences in skin pH to baseline (day 1) at D15 (day 15), when applied to A=untreated lesion skin or B-treated lesion skin.

FIG. 5 shows a bar chart with mean values of differences in skin hydration to baseline (day 1) at D15 (day 15), when applied to A=untreated lesion skin or B-treated lesion skin.

FIG. 6 shows photographs of an infant patient suffering from atopic dermatitis before the treatment, and 5 days after treatment with an example composition in accordance with the disclosure.

### DETAILED DESCRIPTION

SCORAD (SCORing Atopic Dermatitis) as referred to herein is a well-known clinical tool to assess the extent and severity of eczema. Extent is measured by the affected area as a percentage of the whole body. Severity is determined by the intensity of each of the following signs assessed as absent (0), mild (1), moderate (2) or severe (3): dryness; erythema (i.e., otherwise referred to as redness); edema and papules (i.e., swelling); weeping and crusts (i.e., otherwise referred to as oozing or crusting); excoriation (e.g., skin picking, as determined by scratch marks); and lichenification (i.e., otherwise referred to as skin thickening). Subjective symptoms on pruritus and sleep loss could also be included. (see https://dermnetnz.org/topics/scorad; Journal of Cutaneous Medicine and Surgery 2018, Vol. 22 (1S) 10S-16S “Approach to the Assessment and Management of Adult Patients with Atopic Dermatitis: A consensus Document. Section II: Tools for Assessing the Severity of Atopic Dermatitis” and Dermatology. 1993; 186(1): 23-31. doi: 10.1159/000247298), all of which are incorporated herein by reference in their entirety. Local SCORAD refers to SCORAD assessment applied to the intensity of the signs on one local site or test area on the skin. The intensity scores are then added together to give a score between 0=minimum and 18=maximum. This may otherwise be referred to as a SCORAD intensity score for a particular or local region.

Atopic dermatitis as referred to herein is a skin condition that causes patches of skin that are itchy, cracked and sore. Atopic dermatitis may otherwise be referred to as atopic eczema.

Severe atopic dermatitis as referred to refers to atopic dermatitis characterized by widespread areas of dry skin, incessant itching, redness (with or without excoriation, extensive skin thickening, bleeding, oozing, cracking, and alteration of pigmentation); severe limitation of everyday activities and psychosocial functioning, nightly loss of sleep as may be determined by the United Kingdom's National Institute of Health and Care Excellence. Severe atopic dermatitis may otherwise be characterized by a local SCORAD score of greater than 12.

Moderate atopic dermatitis as referred to herein refers to atopic dermatitis characterized with areas of dry skin, frequent itching, redness (with or without excoriation and localized skin thickening); moderate impact on everyday activities and psychosocial well-being, frequently disturbed sleep), for example, as may be determined by the United Kingdom's National Institute of Health and Care Excellence. Moderate atopic dermatitis may otherwise be characterized as atopic dermatitis with a local SCORAD score of between 6 and 12.

Mild atopic dermatitis as referred to herein may be atopic dermatitis characterized with areas of dry skin, little impact on everyday activities, sleep and psychosocial well-being as may be determined by the United Kingdom's National Institute of Health and Care Excellence. Mild atopic dermatitis may otherwise be referred to as eczema with a local SCORAD score of less than 6.

The composition for use in the present invention is formulated to be suitable for topical administration to skin. Topical administration refers to a method of administration where the composition is directly applied to a particular part of a body surface, such as the skin and more specifically in this context, affected skin.

As referred to herein, “before treatment” is taken to be before the start of treatment, e.g., the measurement just before the first application of the composition on day 1.

The terms “treatment” and “treating” herein refer to an approach for obtaining beneficial or desired results in a subject, which includes a prophylactic benefit and optionally also a therapeutic benefit. In some embodiments, “treatment” refers to a therapeutic benefit.

“Prophylactic benefit” refers to delaying or eliminating the appearance of a disease or condition, delaying or eliminating the onset of symptoms of a disease or condition, slowing, halting, or reversing the progression of a disease or condition, or any combination thereof.

“Therapeutic benefit” refers to eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the patient may still be afflicted with the underlying disorder.

When ranges are used herein, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range may vary. Typical experimental variabilities may stem from, for example, changes and adjustments necessary during scale-up from laboratory experimental and manufacturing settings to large scale.

The features of any dependent claim may be readily combined with the features of any of the independent claims or other dependent claims.

It must be noted that as used herein and in the appended claims, the singular forms “a”, “an”, and “the” include plural referents unless the context clearly dictates otherwise.

Unless defined otherwise, all technical and scientific terms used herein have the same meanings as commonly understood by one of ordinary skill in the art to which this invention belongs. Abbreviations used herein have their conventional meaning within the chemical and biological arts, unless otherwise indicated.

The term “subject” refers to any suitable subject, including any animal, such as a mammal. In an embodiment, the subject is a human. The subject may otherwise be referred to as the patient herein. In some embodiments, the subject is an adult subject (i.e., years 18 or older). In some embodiments, the subject is a paediatric subject (e.g., a subject less than 18 years old, or a subject less than 15 years old, or a subject less than 12 years old, or a subject less than 10 years old, or a subject less than 5 years old).

The term “comprising” (and related terms such as “comprise” or “comprises” or “having” or “including”) includes those embodiments, for example, an embodiment of any composition of matter, composition, method, or process, or the like, that “consist of” or “consist essentially of” the described features. The term “comprises” or “comprising” can be used interchangeably with “includes”.

### Composition for Use in a Method of Treating/Methods of Treating

In one or more of the above aspects, the composition disclosed herein is used for the treatment of atopic dermatitis. The atopic dermatitis may be mild, mild to moderate, moderate, moderate to severe or severe atopic dermatitis. In preferred embodiments, the atopic dermatitis is moderate atopic dermatitis, or the atopic dermatitis has a local SCORAD score of at least 6, or between 6 and 12 at the start of treatment.

In one or more of the aspects described herein, the patient is preferably human. In some embodiments, the human is an infant, adolescent or adult.

In one or more of the above aspects, the composition is applied at least two times a day. In some embodiments, the composition is applied twice daily. In some embodiments, the composition is applied every 6 to 18 hours, or every 8 to 14 hours, or about every 12 hours. In some embodiments, the composition is applied twice daily, or at least 3 times a day, or at least 4 times a day, or at least 5 times a day, or at least 6 times a day, or at least 7 times a day, or at least 8 times a day, or at least 9 times a day, or at least 10 times a day. In preferred embodiments, the composition is applied twice daily. This is more convenient for the patient such that they do not have to repeatedly administer the cream throughout the day.

In one or more of the above aspects, the method of treating dermatitis may not include treatment with a pharmaceutical active (i.e., different to treatment with the composition comprising the polyhydroxy acid and zinc salt of its conjugate base described herein). In preferred embodiments, the method of treating atopic dermatitis is free of steroids or corticosteroids (e.g., the method of treatment is free of hydrocorticosteroid/hydrocortisone treatment or free of prednisolone treatment, or free of prednicarbate treatment, or free of methylprednisolone treatment, or free of triamcinolone treatment, or free of betamethasone treatment, or free of mometasone treatment, or free of clobetasol treatment). In preferred embodiments, the method of treating atopic dermatitis is free of calcineurin inhibitors. In preferred embodiments, the method of treating atopic dermatitis is free of antibodies. In preferred embodiments, the method of treating atopic dermatitis is free of antihistamines. In preferred embodiments, the method of treating atopic dermatitis is free of antibiotics. In preferred embodiments, the method of treatment is free of colloidal oatmeal. In preferred embodiments, the method of treating atopic dermatitis is free of antiseptics. In preferred embodiments, the method of treating atopic dermatitis is free of biologics. In preferred embodiments, the method of treatment is free of antipruritics (e.g., hydrocortisone, menthol, pramoxine HCl, menthoxypropanediol, colloidal oatmeal). In preferred embodiments, the method of treatment is free of lactic acid, PCA (Pyrrolidone Carboxylic acid) or urea. Even in the absence of additional pharmaceutical actives including steroids, (e.g. hydrocortisone), the composition for use of the present invention can significantly alleviate the symptoms of atopic dermatitis, e.g., by reducing the local SCORAD score significantly even after a short timescale. This can be achieved without the presence of other pharmaceutical actives which may be harsher, have increased side-effects and be more expensive as compared to use of the composition described herein. As referred to herein, calcineurin inhibitors is any drug which inhibits the action of calcineurin. Calcineurin is an enzyme that activates T-cells of the immune system. In some embodiments, the calcineurin inhibitors are pimecrolimus or tacrolimus.

In one or more of the above aspects, the composition may be topically applied for any suitable length of time. In one or more of the above aspects, the composition may be topically applied for up to 28 days (i.e., less than or equal than 4 weeks), or up to 25 days, or up to 22 days, or up to 20 days, or up to 19 days, or up to 18 days, or up to 17 days, or up to 16 days, or up to 15 days, or up to 14 days. In some embodiments, the composition may be topically applied for at least 5 days, or at least 8 days, or at least 10 days, or at least 11 days, or at least 12 days, or at least 13 days, or at least 14 days. In some embodiments, the composition may be topically applied for 5 days up to 28 days, or for 10 days up to 28 days, or 12 days up to 20 days, or 14 days up to 18 days, or 12 days up to 16 days, or about 14 days. A shorter treatment period which significantly alleviates symptoms is preferable for the patient.

In one or more of the above aspects, the composition may be topically applied in an amount of 0.25 g to 5 g, or about 0.5 g to 3 g, or about 0.75 g to 2 g. The amount of composition applied may depend on the size of the lesion. In some embodiments, the amount of composition may be applied in an amount of 1 fingertip unit (FTU) to 7 FTU (where 1 FTU corresponds to 0.5 g), or about 2 FTU, or about 3 FTU, or about 4 FTU, or about 5 FTU, or about 6 FTU. Depending on the age/size of the subject and the area of skin to be treated, the recommended dosage will vary (e.g., for an adult subject a dosage the dosage may be 1 FTU for a hand, 1 FTU for an elbow, 1 FTU for a knee, 1 FTU for a foot, 2.5 FTU for face and back of neck, 4 FTU for an arm, 6 FTU for a leg, 7 FTU for chest and belly, 7FTU for back and buttocks; whereas for a child subject the dosage may be between 1 and 2 FTU for the face and back of neck, between 1 and 2.5 FTU for an arm and a hand, between 1.5 and 4.5 FTU for a leg and a foot, between 1 FTU and 3.5 FTU for the chest and belly, and between 1.5 FTU and 5 FTU for the back and buttocks). In some embodiments, the composition applied is less than 500 g per week, or less than 400 g per week, or less than 300 g per week.

In one or more of the above aspects, the composition may be topically applied to one or more of the face, neck, leg, foot, chest/belly, back, buttocks, hand, elbow, knee, genitals, or foot.

In one or more of the above aspects, the local SCORAD score of the atopic dermatitis at the start of treatment is at least 6, or at least 7, or at least 8, or at least 9, or at least 10, or at least 11, or at least 12. In some embodiments, the local SCORAD score of the atopic dermatitis is less than 12. In some embodiments, the local SCORAD score of the atopic dermatitis is less than 11, or less than 10, or less than 9, or equal to or less than 8. In some embodiments, the local SCORAD score of the atopic dermatitis is at least 6, or preferably between 6 and 12, or between 6 and 11, or between 6 and 10, or between 6 and 9, or between 6 and 8. In one or more of the above aspects, the local SCORAD score of the atopic dermatitis at the start of treatment is equal to or greater than 6, or 7, or 8, or 9, or 10, 11, or 12.

In one or more of the above aspects, the method of treating involves identifying a patient suffering from moderate atopic dermatitis, e.g., by local SCORAD score and determining that the local SCORAD score is at least 6, for example, between 6 and 12. In some embodiments, the identifying is carried out by a doctor or dermatologist. In some embodiments, after identifying a patient suffering from moderate atopic dermatitis, the method of treating involves prescribing the composition disclosed herein for use. In some embodiments, the method further comprises measuring the local SCORAD score after a time period and determining whether further treatment is needed. In some embodiments, the measuring is carried out by a doctor or dermatologist. In some embodiments, the time period is less than 4 weeks, or less than or equal to 3 weeks, or less than or equal to 2 weeks, for example, 2 weeks.

In one or more of the above aspects, the method of treating involves identifying a patient with atopic dermatitis with a local SCORAD score less than 12, or less than or less than 11, or less than 10, or less than 9, or equal to or less than 8 . . . . In some embodiments, the identifying is carried out by a doctor or dermatologist. In some embodiments, after identifying a patient, the method of treating involves prescribing the composition disclosed herein for use. In some embodiments, the method further comprises measuring the local SCORAD score after a time period and determining whether further treatment is needed. In some embodiments, the measuring is carried out by a doctor or dermatologist. In some embodiments, the time period is less than 4 weeks, or less than or equal to 3 weeks, or less than or equal to 2 weeks, for example, 2 weeks.

In one or more of the above aspects, the method of treating involves identifying a patient with atopic dermatitis with a local SCORAD of at least 6, or between 6 and 12, or between 6 and 11, or between 6 and 10, or between 6 and 8. In some embodiments, the identifying is carried out by a doctor or dermatologist. In some embodiments, after identifying a patient, the method of treating involves prescribing the composition disclosed herein for use. In some embodiments, the method further comprises measuring the local SCORAD score after a time period and determining whether further treatment is needed. In some embodiments, the measuring is carried out by a doctor or dermatologist. In some embodiments, the time period is less than 4 weeks, or less than or equal to 3 weeks, or less than or equal to 2 weeks, for example, 2 weeks.

In one or more of the above aspects, the method of treating may result in a reduction of local SCORAD score (ΔSCORAD) of at least 1, or at least 2, or at least 3, or at least 4 compared to baseline local SCORAD at the start of treatment. In some embodiments, the reduction in local SCORAD score is obtained in less than 4 weeks, or less than or equal to 3 weeks, or less than 18 days, or less than or equal to 2 weeks. In some embodiments, the reduction in local SCORAD is obtained between 5 and up to 28 days, or between 10 and up to 28 days, or between 10 and 25 days, or between 10 and 21 days, or between 12 and 18 days, or between 12 and 16 days. In the examples disclosed herein, the method of treating across an average sample led to a reduction in local SCORAD score of about 4 after 14 days (+2 days).

In one or more of the above aspects, the method of treating may result in a reduction in one or more of a reduction in dryness, erythema, edema and papules, weeping and crusts, excoriation, lichenification score as compared to before treatment. The “score” refers to the score for each of dryness, erythema, edema and papules, weeping and crusts, excoriation, lichenification assessed as part of the local SCORAD assessment, wherein 0=absent, 1=slight, 2=moderate and 3=strong. In some embodiments, the method of treating may result in a reduction of two or more of a reduction in dryness, erythema, edema and papules, weeping and crusts, excoriation, lichenification as compared to before treatment, or three or more, or four or more, or five or more, of a reduction in dryness, erythema, edema and papules, weeping and crusts, excoriation and lichenification as compared to before treatment.

In one or more of the above aspects, the method of treating may result in a reduction in dryness score as compared to before treatment. In one or more of the above aspects, the method of treating may result in a reduction in erythema score as compared to before treatment. In one or more of the above aspects, the method of treating may result in a reduction in edema and papules score as compared to before treatment. In one or more of the above aspects, the method of treating may result in a reduction in weeping and crusts score as compared to before treatment. In one or more of the above aspects, the method of treating may result in a reduction in excoriation score as compared to before treatment. In one or more of the above aspects, the method of treating may result in a reduction in lichenification score as compared to before treatment.

In one or more of the above aspects, the method of treating may result in a reduction in skin pH as compared to before treatment. In some embodiments, the method of treating results in a reduction in skin pH of at least 0.5, or at least 0.6, or at least 0.7, or at least 0.8 as compared to before treatment. In one or more of the above aspects, the method of treating may result in a reduction in skin pH of between 0.5 and 0.8, or between 0.55 and 0.75, or between 0.6 and 0.7 as compared to before treatment. The determination of skin pH may be as determined by skin pH meter.

In one or more of the above aspects, the method of treating may result in an increase in skin hydration as compared to before treatment. The determination of skin hydration may be determined by electrical capacitance method, e.g., using a Corneometer. In some embodiments, the method of treating results in an increase in skin hydration of at least at least 2 a.u. skin capacitance, or at least 3 a.u. skin capacitance, or at least 4 a.u. skin capacitance as compared to before treatment. In some embodiments, the method of treating results in an increase in skin hydration of between 2 and 6 a.u. skin capacitance as compared to before treatment, or between 3 and 5.5 a.u. skin capacitance, or at least 4 and 5 a.u. skin capacitance as compared to before treatment.

### Composition

Quantities expressed herein as % w/w of the composition refer to the total composition unless specified otherwise.

The composition for use of the present invention includes a polyhydroxy acid and a zinc salt of its conjugate base. These may be referred to as the active ingredients of the composition. In certain embodiments, said active ingredients may be in the aqueous-glycol phase of the composition. In some embodiments, the polyhydroxy acid may be selected from lactobionic acid, gluconolactone/gluconic acid or a combination of lactobionic acid and gluconolactone/gluconic acid or salts thereof. In preferred embodiments, the polyhydroxy acid is lactobionic acid or salts thereof. The salts may include one or more of zinc, calcium, potassium, sodium, magnesium, or iron salts. The composition of the present invention comprises zinc ions (Zn²⁺). Zinc ions can provide an antimicrobial and antibacterial effect which is beneficial in the treatment of atopic dermatitis.

In embodiments the polyhydroxy acid is lactobionic acid and the zinc salt of its conjugate base is zinc lactobionate, and the composition comprises a mixture of zinc lactobionate and lactobionic acid. In embodiments the composition comprises a mixture of zinc lactobionate and lactobionic acid in a ratio of about 1:2 to 1:6, or about 1:4. In the examples, the zinc salt derives from zinc oxide (i.e., zinc oxide is used as the ingredient from which the zinc salt is formed). The zinc salt may derive from any suitable zinc-containing compound. However, it is preferred that the composition for use in the present invention does not comprise or use as an ingredient zinc pyrithione (i.e., which is sometimes used in unrelated anti-dandruff shampoo formulations).

The amount of polyhydroxy acid used in manufacturing the composition may comprise 2.0-10.0% w/w of the composition. The amount of zinc oxide used in manufacturing the composition may be greater than 0.05% w/w, or at least about 0.1% w/w. Zinc oxide ingredient may be used in an amount from 0.05-0.5% w/w, 0.05-0.2% w/w and preferably about 0.1% w/w.

In preferred embodiments, the composition does not include any further actives or medicaments. In preferred embodiments, the composition is free of steroids (e.g., hydrocortisone). In preferred embodiments, the composition is free of calcineurin inhibitors. In preferred embodiments, the composition is free of colloidal oatmeal. In preferred embodiments, the composition is free of antibodies. In preferred embodiments, the composition is free of antihistamines.

The composition of the present invention may comprise 2.0-10.0% w/w (percent weight by weight) of the composition of the polyhydroxy acid and the zinc salt of its conjugate base, or from 3.0-8.0% w/w, or from 4-6.0% w/w of the composition of the polyhydroxy acid and the zinc salt of its conjugate base. The composition of the present invention may comprise the polyhydroxy acid and the zinc salt of its conjugate base at greater than or equal to 2.5, 3.0, 3.5, 4.0, 4.5 or 5.0% w/w of the composition. The composition of the present invention may comprise the polyhydroxy acid and the zinc salt of its conjugate base at less than or equal to 9.5, 9.0, 8.5, 8.0, 7.5%, 7%, 6.5%, 6% or 5.5% w/w of the composition. The amount of the polyhydroxy acid in the composition of the present invention may be from about 2.0 to 10% w/w, 3.0 to 6.5, or from 3.5 to 6.0, or from 4.0 to 5.5 or is about 5% w/w of the composition.

The composition of the present invention can further comprise water, and the composition can comprise from 30-60% w/w water, or from 35%-60% w/w water or from 40%-60% w/w water, or from 40-50% w/w water, for example 44-46% w/w water. In some embodiments, the composition of the present invention comprises greater than 40% w/w water, or greater than 41% w/w water, or greater than 42% w/w water, or greater than 43% w/w water, or greater than 44% w/w water. In some embodiments and examples, the water is deionised water (e.g., where no salt is dissolved in the water). In embodiments, a salt is dissolved in the water in the composition of the present invention. The salt may comprise a group 1 metal anion, such as a sodium. The salt may comprise a halogen cation, such as chloride. The salt may be sodium chloride. The salt may be present at 0.1-3% or 0.5-2.0% in the water of the aqueous-glycol phase).

The composition of the present invention comprises a partition coefficient enhancer. The partition coefficient enhancer may be in the aqueous-glycol phase of the composition. The partition coefficient enhancer may be selected from propylene glycol, butylene glycol, pentylene glycol, hexylene glycol, 1,5 pentane diol, or a combination thereof. In preferred embodiments, the partition coefficient enhancer is butylene glycol. Butylene glycol is found to have less association with skin irritation. In alternative examples, the partition coefficient enhancer is propylene glycol. The partition coefficient enhancer may be present in an amount that is 15-30% w/w, 15-25% w/w. In some embodiments, the partition coefficient enhancer may be present in an amount of about 15-20% w/w, 16 to 18% w/w or about 17.5% w/w of the composition (e.g., butylene glycol).

The composition of the present invention may also comprise one or more emollients which may be in the oil phase of the composition. The one or more emollient may be present in an amount that is 10-30% w/w of the composition. In some embodiments, the emollient is a high molecular weight hydrocarbon, preferably selected from mineral oil, petrolatum, paraffin, and mixtures thereof. The emollient may be selected from hydrogenated polydecene, hydrogenated didecene, hydrogenated polyisobutene and mixtures thereof. In embodiments the emollient is hydrogenated polydecene. The composition of the present invention can comprise from 10 to 30% w/w, 10 to 25% w/w, 10 to 19% w/w, from 12 to 16% w/w or about 15% w/w of an emollient.

The composition of the present invention comprises a diffusion coefficient enhancer which is selected from a C12 to C14 straight chain fatty acid or a C14 straight chain primary alcohol. The diffusion coefficient enhancer may be in the oil phase of the composition. The diffusion coefficient enhancer may be 1-tetradecanol or myristic acid. In embodiments the diffusion coefficient enhancer is 1-tetradecanol. 1-tetradeconol is otherwise known as myristryl alcohol. The diffusion coefficient enhancer can be present in an amount that is 1.0-2.0% w/w or 1.2-2.0% w/w, 1.2-1.8% w/w or about 1.5% w/w of the composition. In some embodiments, the diffusion coefficient enhancer is present in an amount that is greater than 1.2% w/w, or greater than 1.4% w/w, or greater or equal to 1.5% w/w of the composition. The diffusion coefficient enhancer is generally at saturation or close to saturation (i.e., at least 95% saturation).

The composition of the present invention has a low or an acidic pH. The composition of the present invention has a pH in the range of 2.7-5.0. More preferably, the pH of the composition is less than 5.0, or less than 4.5, or less than 4.0, or less than 3.5. The composition may have a pH in the range of 3.0 to 4.5, or in the range of 3.0 to 4.0, or in the range of 3.0 to 3.5, or about 3.2. The composition of the present invention may comprise an acid or base to modulate the pH of the composition. An acid that can be used to adjust composition pH is citric acid. A base that can be used is sodium hydroxide. In some examples, the composition comprises 10% sodium hydroxide solution. In some embodiments, the composition comprises about 0.2% w/w of 10% sodium hydroxide solution. Different to previous compositions, a pH of between 3.0 to 3.5, (e.g., about 3.2) enables the composition was found to have a more sustained skin pH/lowering capacity over a 12 hour period, as compared to compositions of a higher pH (e.g., pH 4.0). pH described herein refers to the pH of the whole composition. The pH may be determined using standard methods, for example, using a pH meter.

The composition of the present invention may also comprise one or more surfactants and/or cosurfactants. The composition may comprise 3-15% w/w, 6-14% w/w, 8-13.5% w/w, or 9-13% w/w or about 11% w/w of one or more surfactants and/or co-surfactants. The surfactants stabilise the viscosity or the interface of the aqueous-glycol and oil phases. The composition may comprise a primary surfactant or a primary surfactant system.

The composition may comprise 2-10% w/w, 2.5-9% w/w or from about 3 to 8% w/w, or from about 5 to 7% w/w, or about 6% w/w, of a primary surfactant or primary surfactant system. A primary surfactant system may comprise methyl glucose sesquistearate (Glucamate SS) and a PEG methyl glucose sesquistearate, e.g., PEG-20 methyl glucose sesquistearate (Glucamate SSE-20). In some embodiments, the composition may comprise methyl glucose sesquistearate and a PEG methyl glucose sesquistearate (e.g., PEG-20 methyl glucose sesquistearate) in a ratio of 5:1 to 1:5, more preferably, from 5:1 to 1:1, or about 3:1.

In an embodiment, the composition may comprise 2.5-7% w/w methyl glucose sesquistearate, or about 3 to 6% w/w methyl glucose sesquistearate, or about 4.5% w/w methyl glucose sesquistearate. In some embodiments, the composition may comprise at least 1% w/w methyl glucose sesquistearate, or at least 2% w/w, or at least 3% w/w, or at least 4% w/w. In an embodiment, the composition may comprise 0.5-4% w/w PEG-20 methyl glucose sesquistearate, or from 1 to 2% w/w PEG-20 methyl glucose sesquistearate. In some embodiments, the composition may comprise less than 2% w/w PEG-20 methyl glucose sesquistearate.

The composition may further comprise a co-surfactant, which may be otherwise referred to as a secondary surfactant. The co-surfactant can be a C16 to C22 alcohol, more preferably a C20 to C22 alcohol. In some embodiments, the C16 to C22 alcohol is behenyl alcohol. In some examples, the composition may comprise 2.0-5.5% w/w, 2.5-7.5% w/w or from about 4-6% w/w, or from about 4.5-6% w/w, or about 5% w/w co-surfactant. In some embodiments, the co-surfactant is behenyl alcohol.

The composition may comprise one or more of a ceramide, a cholesterol and a fatty acid. In some embodiments, the composition may comprise a ceramide. In some embodiments, the ceramide is present in an amount between 0.4 to 2% w/w, or about 0.5 to 1.5% w/w, or about 0.75 to 1.25% w/w, or about 1.0% w/w of the composition.

In some embodiments, the composition may comprise a fatty acid. The fatty acid may be a polyunsaturated fatty acid. In some embodiments, the fatty acid may be a C16 to C20 fatty acid, for example, a C18 fatty acid. In some examples, the fatty acid is linoleic acid. In some embodiments, the linoleic acid is present in an amount between 0.2 and 0.5% w/w of the composition, or between 0.3 and 0.4% w/w of the composition, preferably about 0.33% w/w.

In some embodiments, the composition may comprise cholesterol. In some embodiments, the cholesterol is present in an amount between 0.2 and 0.5% w/w of the composition, or between 0.3 and 0.4% w/w of the composition, preferably about 0.33% w/w.

In some examples, the fatty acid may be linoleic acid. In embodiments the composition comprises a ceramide, a cholesterol and a fatty acid in the ratio of approximately 3:1:1. In embodiments the fatty acid may be linoleic acid. These components may act as a skin barrier restoration system.

The composition of the present invention may further comprise a PEG copolymer. In some examples, the PEG copolymer is a PEG/HDI Copolymer Bis-Decyltetradeceth-20 Ether. In some examples, the PEG is PEG 240. This may behave as a viscosity modifier. In some embodiments, the composition further comprises a C12-C14 ether. In some embodiments, the C12-C14 ether is a PEG ether. In some embodiments, the C12-C14 ether comprises the PEG ether of tridecyl alcohol. This may behave as a viscosity modifier.

In some embodiments, the composition further comprises acrylates/C10-C30 an alkyl acrylate cross-polymer. This may behave as a viscosity modifier.

In some embodiments, the composition comprises PEG copolymer is a PEG/HDI Copolymer Bis-Decyltetradeceth-20 Ether (e.g., a PEG-240/HDI Copolymer Bis-Decyltetradeceth-20 Ether), a PEG ether of tridecyl alcohol and acrylate/C10-C30 alkyl acrylate cross-polymer. This may behave together as viscosity modifiers and are found to improve the texture and stability of the composition.

The composition for use in the present invention may further comprise a stabilizer, a preservative, a salt or a buffer. The aqueous-glycol phase may comprise a stabilizer. Stabilizers include a xanthan gum or a crystal growth inhibitor. In some embodiments, the composition for use is free from xanthan gum or crystal growth inhibitor.

In some embodiments, the composition for the use according to the preceding claims, comprises
4-6% w/w polyhydroxy acid and zinc salt of its conjugate base, wherein the polyhydroxy acid is lactobionic acid
15-20% w/w partition coefficient enhancer, preferably butylene glycol
1-2% w/w diffusion coefficient enhancer, preferably tetradecyl alcohol, and
10-20% w/w emollient, preferably hydrogenated polydecene
3-12% w/w surfactant (i.e., primary surfactant), preferably wherein the surfactant comprises one or more of methyl glucose sesquistearate, PEG-20 methyl glucose sesquistearate, glyceryl stearate or a combination thereof, preferably comprising 3-6% w/w methyl glucose sesquistearate and 1-2% w/w PEG-20 methyl glucose sesquistearate
40-60% w/w water, and
up to 3% w/w humectant, preferably glycerol, and
optionally a co-surfactant, preferably in an amount up to 6% w/w of the composition
optionally a ceramide, preferably in an amount up to 1.5% w/w of the composition
optionally cholesterol, preferably in an amount up to 0.5% w/w of the composition
optionally a fatty acid, preferably in an amount up to 0.5% w/w of the composition
optionally a PEG copolymer, preferably in an amount up to 1% w/w of the composition
optionally a acrylates/C10-C30 alkyl acrylate copolymer, preferably in an amount up to 0.5% w/w of the composition, and
optionally a preservative, preferably in an amount up to 1% w/w of the composition wherein the pH of the composition is between 3.0 and 3.5.

In some embodiments, the composition for the use according to the preceding claims, comprises
4-6% w/w polyhydroxy acid and zinc salt of its conjugate base, wherein the polyhydroxy acid is lactobionic acid
15-20% w/w butylene glycol
1-2% w/w diffusion coefficient enhancer, preferably tetradecyl alcohol, and
10-19% w/w emollient, preferably hydrogenated polydecene
3-6% w/w methyl glucose sesquistearate and
1-2% w/w PEG-20 methyl glucose sesquistearate
40-60% w/w water, and
up to 3% w/w humectant, preferably glycerol, and
optionally a co-surfactant, preferably in an amount up to 6% w/w of the composition
optionally a ceramide, preferably in an amount up to 1.5% w/w of the composition
optionally cholesterol, preferably in an amount up to 0.5% w/w of the composition
optionally a fatty acid, preferably in an amount up to 0.5% w/w of the composition
optionally a PEG copolymer, preferably in an amount up to 1% w/w of the composition
optionally a acrylates/C10-C30 alkyl acrylate copolymer, preferably in an amount up to 0.5% w/w of the composition, and
optionally a preservative, preferably in an amount up to 1% w/w of the composition wherein the pH of the composition is between 3.0 and 3.5.

### Embodiments of the Present Invention Include

A. A composition for use in a method of treating moderate atopic dermatitis,
wherein the composition is topically applied on the skin at least 2 times a day,
wherein the composition has a pH in the range of 2.7 to 5.0 and comprises
a polyhydroxy acid and a zinc salt of its conjugate base, wherein the polyhydroxy acid is selected from lactobionic acid, gluconolactone, or a combination of lactobionic acid and gluconolactone,
a partition coefficient enhancer selected from propylene glycol, butylene glycol, pentylene glycol, hexylene glycol, 1,5 pentane diol or a combination thereof,
a diffusion coefficient enhancer which is selected from a C12 to C14 straight chain fatty acid or a C14 straight chain primary alcohol.

B. A method of treating moderate atopic dermatitis, wherein the method comprises topically applying the composition on the skin at least 2 times a day for between 10 and up to 28 days, wherein the composition has a pH in the range of 2.7 to 5.0 and comprises
a polyhydroxy acid and a zinc salt of its conjugate base, wherein the polyhydroxy acid is selected from lactobionic acid, gluconolactone, or a combination of lactobionic acid and gluconolactone,
a partition coefficient enhancer selected from propylene glycol, butylene glycol, pentylene glycol, hexylene glycol, 1,5 pentane diol or a combination thereof,
a diffusion coefficient enhancer which is selected from a C12 to C14 straight chain fatty acid or a C14 straight chain primary alcohol.

C. A composition for use in a method of treating atopic dermatitis, wherein the composition is topically applied on the skin for between 10 and up to 28 days at least 2 times a day, wherein the composition has a pH in the range of 2.7 to 5.0 and comprises
a polyhydroxy acid and a zinc salt of its conjugate base, wherein the polyhydroxy acid is selected from lactobionic acid, gluconolactone, a combination of lactobionic acid and gluconolactone,
a partition coefficient enhancer selected from propylene glycol, butylene glycol, pentylene glycol, hexylene glycol, 1,5 pentane diol or a combination thereof,
a diffusion coefficient enhancer which is selected from a C12 to C14 straight chain fatty acid or a C14 straight chain primary alcohol.

D. A method of treating atopic dermatitis, wherein the method comprises topically applying the composition on the skin for between 10 and up to 28 days at least 2 times a day, wherein the composition has a pH in the range of 2.7 to 5.0 and comprises
a polyhydroxy acid and a zinc salt of its conjugate base, wherein the polyhydroxy acid is selected from lactobionic acid, gluconolactone, a combination of lactobionic acid and gluconolactone,
a partition coefficient enhancer selected from propylene glycol, butylene glycol, pentylene glycol, hexylene glycol, 1,5 pentane diol or a combination thereof,
a diffusion coefficient enhancer which is selected from a C12 to C14 straight chain fatty acid or a C14 straight chain primary alcohol.

E. The composition for the use according to embodiments C or D, wherein the atopic dermatitis is moderate or mild atopic dermatitis

F. The composition for the use according to any of the preceding embodiments, wherein the composition is applied twice daily.

G. The composition for the use according to any of the preceding embodiments, wherein the composition is free of steroids, topical calcineurin inhibitors.

H. The composition for the use according to the preceding embodiments, wherein the method of treating atopic dermatitis does not include treatment with a steroid, topical calcineurin inhibitors or other pharmaceutical active.

I. The composition for the use according to any of the preceding embodiments wherein the composition is topically applied for 12 days and 20 days, for example, about 14 days.

J. The composition for the use according to any of the preceding embodiments, wherein the local SCORAD score of the atopic dermatitis at the start of treatment is at least 6, preferably between 6 and 12, for example, between 6 and 10.

K. The composition for the use according to any of the preceding embodiments, wherein the method involves identifying a patient suffering from moderate atopic dermatitis by measuring the local SCORAD score, determining that the local SCORAD score is at least 6, more preferably between 6 and 12.

L. The composition for the use according to embodiment K, wherein the method further comprises measuring the local SCORAD score after a time period to ensure whether further treatment is needed.

M. The composition for the use according to embodiment L, wherein the time period is less than 4 weeks, or less than or equal to 3 weeks, or less than or equal to 2 weeks.

N. The composition for the use according to the preceding embodiments, wherein the method of treating results in a reduction of local SCORAD score (ΔSCORAD) of at least 1, or at least 2, or at least 3, or at least 4 compared to baseline local SCORAD at the start of treatment.

O. The composition for the use according to embodiment N, wherein the reduction in local SCORAD score is obtained in less than 4 weeks, or less than or equal to 3 weeks, or less than or equal to 2 weeks.

P. The composition for the use according to any of the preceding embodiments, wherein the pH of the composition is between 3.0 to 4.0, more preferably from 3.0 to 3.5, and even more preferably about 3.2

Q. The composition for the use according to any of the preceding embodiments, wherein the partition coefficient enhancer is butylene glycol.

R. The composition for the use according to any of the preceding embodiments, wherein the partition coefficient enhancer is present in an amount that is 15-30% w/w of the composition, or from 15-25% w/w of the composition, or from 15-20% w/w of the composition.

S. The composition for the use according to any of the preceding embodiments, wherein the diffusion coefficient enhancer is present in an amount that is 1.0-2.0% w/w of the composition, 1.2-1.8% w/w or about 1.5% w/w of the composition.

T. The composition for the use according to the preceding embodiments, further comprising a ceramide, preferably wherein the ceramide is present in an amount from 0.5 to 1.5% w/w of the composition.

U. The composition for the use according to any of the preceding embodiments, wherein the composition comprises from 3-6% w/w methyl glucose sesquistearate and 1-2% w/w PEG-20 methyl glucose sesquistearate.

V. The composition for the use according to any of the preceding embodiments, further comprising a PEG-240/HDI copolymer bis-decyltetradeceth-20 ether

W. The composition for the use according to the preceding embodiments, further comprising an acrylates/C10-C30 alkyl acrylate copolymer.

X. The composition for the use according to the preceding embodiments, comprising 4-6% w/w polyhydroxy acid and zinc salt of its conjugate base, wherein the polyhydroxy acid is lactobionic acid
15-20% w/w partition coefficient enhancer, preferably butylene glycol
1-2% w/w diffusion coefficient enhancer, preferably tetradecyl alcohol, and
10-20% w/w emollient, preferably hydrogenated polydecene
3-12% w/w surfactant, preferably wherein the surfactant comprises one or more of methyl glucose sesquistearate, PEG-20 methyl glucose sesquistearate, glyceryl stearate or a combination thereof,
40-60% w/w water, and
up to 3% w/w humectant, preferably glycerol, and
optionally a co-surfactant, preferably in an amount up to 6% w/w of the composition
optionally a ceramide, preferably in an amount up to 1.5% w/w of the composition
optionally cholesterol, preferably in an amount up to 0.5% w/w of the composition
optionally a fatty acid, preferably in an amount up to 0.5% w/w of the composition
optionally a PEG-240/HDI copolymer bis-decyltetradeceth-20 ether, preferably in an amount up to 1% w/w of the composition
optionally a acrylates/C10-C30 alkyl acrylate copolymer, preferably in an amount up to 0.5% w/w of the composition, and
optionally a preservative, preferably in an amount up to 1% w/w of the composition
wherein the pH of the composition is between 3.0 and 3.5

### Examples

### Example 1: Trial Regarding the Treatment of Moderate Atopic Dermatitis

### Materials and Methods

TABLE 1
Test Materials

| Code/proDERM | Treatment/Code/Sponsor/Concentration |
|---|---|
| A | Lesional skin, Untreated |
| B | Lesional skin, Treated with Emollient cream |
| | (LCP010-T13R2) |
| Date Samples Received | Nov. 5, 2020 |
| Storage Conditions | Room temperature |
| Type of Product | Emollient cream |
| Study Design | Exploratory |
| | Randomized |
| | Intra-Individual Comparison (before and after treatment) |
| | Controlled (treated versus untreated) |
| Type of Statistics | Comparison between treatments (treated vs. untreated on |
| | lesional skin) |
| Assessment Times | Baseline (Day 1, before test product application |
| | Day 15 |
| Test Area | Body, two skin areas with comparable slight lesions of atopic |
| | dermatitis (specified by the dermatologist, local SCORAD of |
| | at least 6 (mean over two lesions)). Non-lesional areas were |
| | located in a distance of at least 6 cm to lesional skin |
| Assignment of Test | Treatments were randomly assigned to the left and right sides |
| Areas | of the body (see FIG. 1). The assignment was balanced in |
| | accordance to the local SCORAD using a randomization tool |
| | of the eCRF system (SecuTrial ®). The program determines for |
| | each subject if the right or left lesion has a higher score or if |
| | both are equally scored and then assigns the treatments to the |
| | sides in a balanced way |
| Application Volume | According to normal use conditions. Typically about 1 g per |
| | forearm. |
| Application Mode | The correct amount of the product to be applied was |
| | demonstrated by a technician during the first application on |
| | Day 1. The test material(s) were then applied twice daily in |
| | the morning and in the evening for 2 weeks by the subjects at |
| | home, according to application training, on the lesion on the |
| | side to be treated |

### Composition Used in Example 1 Study

| | LCP010-T13 |
|---|---|
| INGREDIENTS | % w/w |
| Zinc Oxide | 0.10 |
| Lactobionic Acid | 4.92 |
| DI Water | 44.57 |
| Butylene Glycol | 17.50 |
| PEG-240/HDI Copolymer Bis-Decyltetradeceth-20 | 0.80 |
| Ether & Trideceth-6 (Avalure Flex-6 CC Polymer) | |
| Glycerol | 2.00 |
| Ethylhexylglycerin | 0.50 |
| Hydrogenated Polydecene | 15.00 |
| Methyl Glucose Sesquistearate (Glucate SS) | 4.50 |
| PEG-20 Methyl Glucose Sesquistearate | 1.50 |
| (Glucamate SSE-20) | |
| Myristyl Alcohol | 1.50 |
| Behenyl Alcohol | 5.00 |
| Ceramide | 1.00 |
| Cholesterol | 0.33 |
| Linoleic acid | 0.33 |
| Acrylates/C10-30 Alkyl Acrylate | 0.25 |
| Crosspolymer (Pemulen EZ-4U) | |
| Sodium Hydroxide solution (10%) (C) | 0.20 |
| TOTAL | 100.00 |
| Fresh cream pH range | 3.1-3.3 |

Different to previously disclosed example compositions, the composition for use in this study comprised butylene glycol as the partition coefficient enhancer because butylene glycol was found to have less association with skin irritation and more suited for very young children.

Also, different to previously disclosed example compositions, is a higher amount of Methyl Glucose Sesquistearate, and a comparatively lower amount of PEG-20 Methyl Glucose Sesquistearate.

Further, the present composition comprises further components aimed to improve the viscosity of the composition, including a PEG/HDI copolymer, Trideceth-6 and an acrylate/C10-30 alkyl acrylate copolymer. These components were found to enhance the stability and texture of the formula as compared to previous compositions.

Different to previous compositions, a pH of between 3.2 enables the composition was found to have a more sustained skin pH/lowering capacity over a 12 hour period, as compared to compositions of a higher pH (e.g., pH 4.0).

### Subjects

All below mentioned inclusion, exclusion criteria and instructions for the subjects were checked by a questionnaire before the start of the study and during the study. Conditions developing during the course of the study listed in the exclusion criteria and instructions, as well as protocol deviations, did not necessarily lead to the subject's exclusion.

### Inclusion Criteria

Written Informed Consent of the subject, or, for underage subjects (<16 years), their parents/legal guardians to let the child participate in the study
Willingness to actively participate in the study and to come to the scheduled visits.
Willingness of the parents/legal guardians to actively support the participation in the study and to come to the scheduled visits with their children
Female or male
From 4 to 65 years of age (without any fixed ratio of age groups)·
Subjects with atopic dermatitis with at least two comparable lesions (at least a local SCORAD of 6 as mean over the two lesions), diagnosed by a dermatologist, ideally in contralateral position (in single cases, this may not be the case)

### Exclusion Criteria

Female subjects: Pregnancy or lactation
Drug addicts, alcoholics.
AIDS, HIV-positive or infectious hepatitis if known to the subjects.
Conditions which exclude a participation or might influence the test reaction/evaluation
Participation or being in the waiting period after participation in similar cosmetic and/or pharmaceutical studies
Active skin disease at the test area (except atopic dermatitis).
One of the following illnesses that might require regular systemic medication: insulin-dependent diabetes, cancer, rheumatic disease
Documented allergies to cosmetic products and/or ingredients
Moles, tattoos, scars, irritated skin, hairs, etc. at the test area that could influence the investigation
Regular use of tanning beds within the last 2 years.
Systemic therapy with immuno-suppressive drugs (e.g. corticosteroids) and/or antihistamines (e.g. antiallergics) within the last 30 days prior to the start of the study.
Topical corticosteroids at the test area within the last 2 weeks prior to the start of the study
Supporting therapy against atopic dermatitis (UV therapy, probiotic homeopathy etc.) within the last 2 weeks prior to the start of the study.
Antiseptic or antibacterial wash or topical products within four weeks prior to start of the study

### Instructions for Trial Participants Prior to the Start of the Study:

The subjects or subjects' parents/legal guardians were instructed not to . . .
smoke within the last 2 hours prior to the instrumental measurements
drink any caffeinated beverages (e.g., coffee, cola) within the last 2 hours prior to the instrumental measurements
apply any leave on cosmetics (e.g., creams, lotions, oily cleansing products) in the test area within the last 7 days prior to the start of the study
apply any detergents (e.g., soaps, shampoos, bath and shower products) in the test area 24 hours before of their scheduled visits

The subjects or subjects' parents/legal guardians were instructed to . . .
wash the test areas only with water 24±2 hours prior to the start of the study
avoid contact of the test area with water (e.g., no bathing, no swimming, no showering) within the last 24 hours prior to the instrumental measurements

### Instructions for Trial Participants Throughout the Course of the Study:

The subjects or subjects' parents/legal guardians were instructed not to:
smoke within the last 2 hours prior to the instrumental measurements
drink any caffeinated beverages (e.g., coffee, cola) within the last 2 hours prior to the instrumental measurements
apply any products with the same indication as the test product in the test area throughout the entire course of the study
apply any leave on cosmetics (e.g., creams, lotions, oily cleansing products) in the test area throughout the entire course of the study
apply any detergents (e.g., soaps, shampoos, bath and shower products) in the test area within 24 hours before the scheduled visit
use any antiseptic/antibacterial wash or topical products throughout the entire course of the study

The subjects or subjects' parents/legal guardians were instructed to
wash the test areas only with water 24±2 hours prior to the scheduled visit.
avoid contact of the test area with water (e.g., no bathing, no swimming, no showering) within the last 24 hours prior to the instrumental measurements.
apply the test product(s) in the test area with the following frequency: 2× daily (in the morning and the evening) throughout the entire course of the study.

The test area is the one, randomly chosen atopic lesion and the surrounding, non-lesional skin (e.g., the arm or leg).
apply the test product(s) in the test area the last time within 3-12 hours before the last study visit
continue to use the normally used detergents (e.g., soaps, shampoos, bath and shower products) in the test area, but not to change the brand or use new products throughout the entire course of the study
refrain from any sun exposure, UV-therapy and/or artificial tanning in the test area throughout the entire course of the study

TABLE 2
Test Schedule

| Day 1 (Baseline) | Day 2 to 14 | Day 15 |
|---|---|---|
| Informed Consent | X | |
| In-/Exclusion Criteria | X | |
| Dermatological | X | X |
| Evaluation | | |
| (SCORAD) | | |
| (by a dermatologist) | | |

### Climatic Conditions

The instrumental measurements took place in an air-conditioned room at a temperature of 22±2° C. and at 50±7.5% relative humidity. Before measurements, the subjects stayed in the climatized room for at least 30 minutes.

### Description of Test Procedure

Day 1: The subjects came to the Study Site. They were informed about the study and gave their written consent. The assessment of local SCORAD was performed by the dermatologist and localization of the lesions (arms or legs).

The test product(s) were issued to the subjects with instructions to use them on the assigned test areas. The assignment of test product(s) was done according to a randomization scheme. The first product application was performed under the guidance of a technician. Further application(s) were performed by the subjects at home.

Day 1 to 14: Subjects used the test product(s) as described above (see ‘Application Mode’).

The last product application was performed within 3-12 hours before the next visit at the Study Site.

Day 15: Subjects returned to the Study Site. After acclimation for at least 30 minutes, the assessment of local SCORAD by dermatologist.

A deviation of ±2 days was accepted, since no substantial influence on the outcome of the study was expected.

Investigational Methods The following investigational method(s) were performed:

### Visual Evaluation

Local SCORAD (SCORing Atopic Dermatitis): At the above-mentioned assessment time(s) the objective skin status on the atopic lesions (erythema, edema and papules, weeping and crusts, excoriation, lichenification, dryness*) were evaluated according to the following scale:
0=Absent
1=Slight
2=Moderate
3=Strong
*Dryness was evaluated on the non-lesional areas.

The local SCORAD per test area is the sum of all scores for all six above mentioned parameters (minimum=0, maximum=18). The objective evaluation was performed by a dermatologist or trained physician. Scores were directly entered into a PC system with an appropriate computer program.

This is in accordance with established and well-known protocols, e.g., as outlined in Dermatology. 1993; 186(1): 23-31. doi: 10.1159/000247298

### Data Listing

Raw data of all parameters and calculated values was listed by parameter, skin area, treatment and assessment times.

Local SCORAD: The sum score for the local SCORAD was calculated as the sum of gradings of the single items mentioned above and was used for further analysis. Further, the differences to Baseline were calculated based on the sum score.

### Descriptive Data Analysis

Local SCORAD:
N, mean, standard deviation, median, minimum, maximum and 95% confidence limits were given for raw data and differences to Baseline for each parameter by treatment and assessment time.
The mean values of raw data and differences to Baseline were presented in bar charts for each parameter by treatment and assessment time with 95% confidence intervals.

Local SCORAD (single items):
N, mean, standard deviation, median, minimum and maximum were given for raw data by treatment, assessment time and item.
Count of single scores and count of scores greater than 0 were presented for raw data per treatment, assessment time and item.

### Statistical Data Analysis

A significance level of 0.05 (alpha) was chosen for statistical analysis. Due to the explorative character of the study, no adjustment for multiplicity was done.

Local SCORAD:
Comparison of treatments (A vs. B) was performed on differences to Baseline by paired t-Test for each assessment time and parameter.

The computation of the statistical data was carried out with commercially available statistics software (SAS for Windows)

### Results

TABLE 3
Demographic Data and Dropouts

| | |
|---|---|
| Subjects Enrolled | 30 |
| Drop Out(s) | 1 without relation to test product(s)* |
| | * Subject #28, for personal reasons |
| Subjects Finished without | 29 |
| Major | |
| Deviations | |
| Complete Data | None |
| Exclusion(s) | |
| Subjects Analyzed | 29* |
| Age: | 30.0 ± 19.4 years (mean ± standard deviation) |
| Sex: | 10 male (34%), 19 female (66%) |

### Local SCORAD

### Local SCORAD by Dermatologist or Adequately Trained Physician

Table 4 presents the mean values and standard deviations of sum scores for local SCORAD at both test areas with lesional skin (untreated (code A) and treated with emollient cream (code B)) by dermatologist and the results of treatment comparison on differences to Baseline by paired t-test. The local SCORAD represents the sum of the scores of all parameters assessed in this evaluation, with the lowest possible value being “0” and the highest possible value “18”. The lower the local SCORAD value, the better was the skin condition.

TABLE 4
Local SCORAD-Mean Values, Standard Deviations, and Comparison
of Treatments on Differences to Baseline by Paired t-Test

| | | | Mean Values ± | | |
|---|---|---|---|---|---|
| | | | SD | | p-values |
| | | | Raw | Diff. | Treat. Comp |
| Time | Code | n | Data | to BL | vs. A |
| BL | A | 29 | 8.2 ± | — | — |
| | | | 2.4 | | |
| | B | 29 | −8.3 ± | — | |
| | | | 2.4 | | |
| D15 | A | 29 | 6.4 ± | −1.8 ± | — |
| | | | 2.6 | 2.3 | |
| | B | 29 | 4.0 ± | −4.3 ± | <0.001 |
| | | | 2.8 | 3.2 | |

bold p-Value: significant (p ≤ 0.05); BL = Baseline
Score: 0 = Minimum possible score; . . . ; 18 = Maximum possible score

The mean values of the local SCORAD decreased for both treatment codes A and B on Day 15 in comparison to Day 1 (Baseline), showing a higher decrease for the treated test area (code B). The comparison of treatments on differences to Baseline unexpectedly showed a statistically significant reduction in mean local SCORAD values for the area treated with the test product (code B) compared to the untreated area.

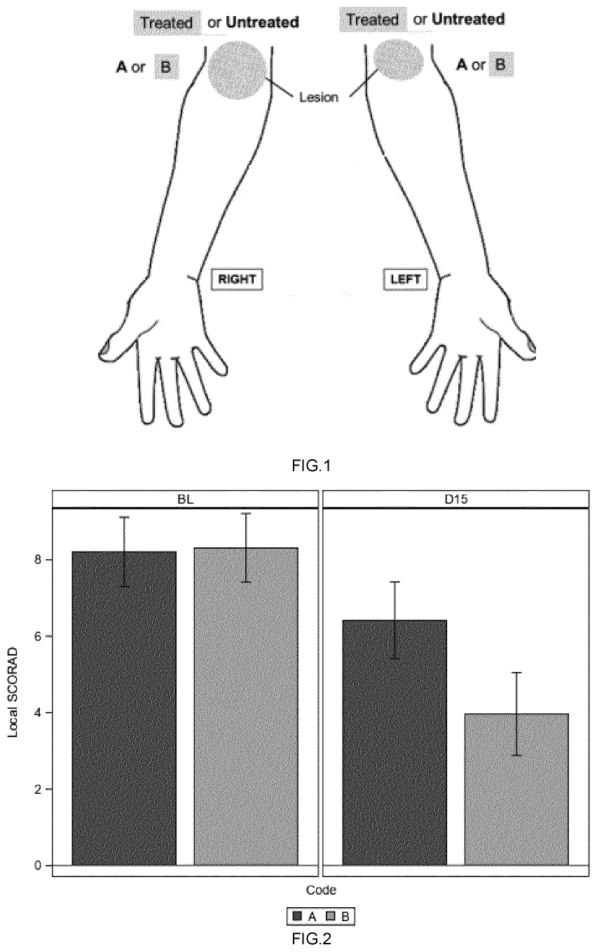

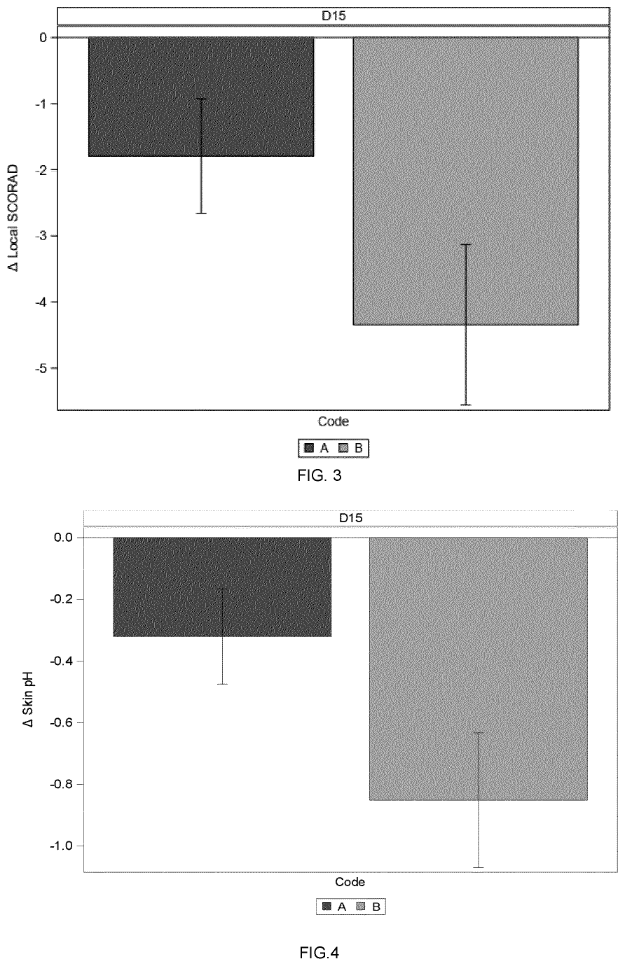

Sum scores and differences to baseline (day 1) of local SCORAD mean values with standard deviations are presented within FIGS. 2 and 3. Count scores for Local SCORAD are also shown in Table 5.

TABLE 5
Counts of Scores for Local SCORAD by Dermatologist or Adequately Trained Physician

| | | | | Absent | Slight | Moderate | Strong | Count > |
|---|---|---|---|---|---|---|---|---|
| Parameter | Time | Code | n | (0) | (1) | (2) | (3) | 0 |
| Dryness | BL | A | 29 | 0 | 2 | 15 | 12 | 29 |
| | | B | 29 | 0 | 3 | 14 | 12 | 29 |
| | D15 | A | 29 | 0 | 8 | 14 | 7 | 29 |
| | | B | 29 | 9 | 12 | 7 | 1 | 20 |
| Erythema | BL | A | 29 | 0 | 11 | 14 | 4 | 29 |
| | | B | 29 | 0 | 11 | 15 | 3 | 29 |
| | D15 | A | 29 | 2 | 15 | 11 | 1 | 27 |
| | | B | 29 | 6 | 18 | 5 | 0 | 23 |
| Edema and | BL | A | 29 | 9 | 10 | 10 | 0 | 20 |
| Papules | | B | 29 | 8 | 11 | 9 | 1 | 21 |
| | D15 | A | 29 | 18 | 6 | 5 | 0 | 11 |
| | | B | 29 | 21 | 6 | 0 | 2 | 8 |
| Weeping and | BL | A | 29 | 16 | 7 | 3 | 3 | 13 |
| Crusts | | B | 29 | 15 | 5 | 7 | 2 | 14 |
| | D15 | A | 29 | 18 | 9 | 2 | 0 | 11 |
| | | B | 29 | 20 | | 1 | 0 | 9 |
| Excoriation | BL | A | 29 | 22 | 3 | 4 | 0 | 7 |
| | | B | 29 | 22 | 4 | 3 | 0 | 7 |
| | D15 | A | 29 | 21 | 5 | 3 | 0 | 8 |
| | | B | 29 | 24 | 4 | 1 | 0 | 5 |
| Lichenification | BL | A | 29 | 2 | 5 | 15 | 7 | 27 |
| | | B | 29 | 1 | 5 | 17 | 6 | 28 |
| | D15 | A | 29 | 0 | 14 | 10 | 5 | 29 |
| | | B | 29 | 6 | 16 | 7 | 0 | 23 |

The difference in score count between D15 and BL for parameters with a count >0 was: Dryness-A=0, B=9; Erythrema-A=2, B=6; Edema and Papules-A=9, B=13; Weeping and Crusts-A=2, B=5; Excoriation-A=−1, B=2; Lichenification-A=−2, B=5.

The difference in score count between D15 and BL for parameters with a count >1: Dryness-A=6, B=18; Erythrema-A=6, B=13; Edema and Papules-A=5, B=8; Weeping and Crusts-A=4, B=8; Excoriation-A=1, B=2; Lichenification-A=7, B=16.

Local SCORAD scores showed an improvement in skin condition at the lesional areas (in terms of the assessed parameters) after 2 weeks of test product application in comparison to baseline values for the untreated as well as for the treated test areas. A significant improvement in skin condition after 2 weeks of test product application was observed in comparison to the untreated test area. The decrease of SCORAD on the untreated areas over time can be explained by changes in single individuals as atopic dermatitis naturally changes over time.

Count scores also show an improvement in dryness, erythema, edema and papules, weeping and crusts, excoriation and lichenification after a 2-week period. Furthermore, in the study, no adverse reactions were observed.

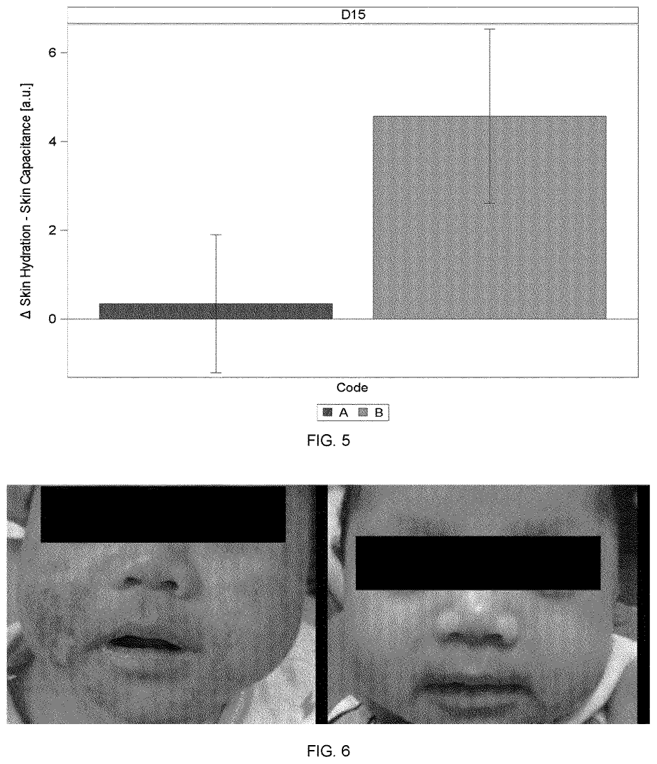

The trial also showed that treatment led to a reduction in skin pH by >0.5 units after 2 weeks (see FIG. 4) and also resulted in a significant increase in skin hydration in comparison to the untreated arm after 2 weeks (see FIG. 5).

### Example 2: Treatment of Composition after 5 Days

The composition used in the treatment of atopic dermatitis was also found to show significant improvement after only 5 days for one infant patient with severe atopic dermatitis.

The composition used was as follows:

| | LCP010 |
|---|---|
| INGREDIENTS | % w/w |
| Zinc Oxide | 0.10 |
| Lactobionic Acid | 4.92 |
| DI Water | 36.42 |
| Propylene Glycol | 17.50 |
| Glycerol | 2.00 |
| Phenoxyethanol | 0.30 |
| Ethylhexylglycerin | 0.20 |
| Hydrogenated Polydecene | 20.00 |
| Methyl Glucose Sesquistearate (Glucate | 0.80 |
| SS) | |
| PEG-20 Methyl Glucose Sesquistearate | 3.20 |
| (Glucamate SSE-20) | |
| Myristyl Alcohol | 1.00 |
| Behenyl Alcohol | 4.00 |
| Ceramide | 1.00 |
| Cholesterol | 0.33 |
| Linoleic acid | 0.33 |
| Polyacrylate-1 Crosspolymer (Carbopol | 4.50 |
| Aqua CC Polymer) | |
| Citric Acid (25%) | 1.50 |
| Sodium Hydroxide solution (10%) | 1.90 |
| TOTAL | 100.00 |

The results can be clearly seen by visual evaluation. FIG. 6 shows the improvement in skin condition after only 5 days, wherein the left photograph shows the skin condition before treatment and the right photograph shows the skin condition after the treatment.

### CONCLUSIONS

The results of the present invention provide compositions for effective use in the treatment of atopic dermatitis, including moderate atopic dermatitis, unexpectedly without the need for combination therapy with other actives, such as steroid treatment. The compositions for use show a significant improvement in local SCORAD scores even after a relatively short 2-week treatment. The present invention therefore provides an improved alternative to steroid treatment, which is associated with many side effects. The present invention is also suitable for use in paediatric patients. The results are surprising since, at the time of filing, although moisturising or emollient only creams are recommended for the management of atopic dermatitis and the prevention of flare-ups in eczema-prone skin, other medicated creams or medications are recommended to be used concurrently especially in moderate and severe atopic dermatitis. Even when used alone, other moisturising creams are recommended for or required a much longer treatment time to observe any meaningful improvement in symptoms.

### Appendix—Raw Data from Example 1

TABLE A1
Descriptive Statistics of Raw Data for Local SCORAD by
Dermatologist or Adequately Trained Physician

| | | | | Mean ± | | | |
|---|---|---|---|---|---|---|---|
| Parameter | Time | Code | n | SD | Median | Min | Max |
| Dryness | BL | A | 29 | 2.3 ± | 2.0 | | 3 |
| | | | | 0.6 | | | |
| | | B | 29 | 2.3 ± | 2.0 | | 3 |
| | | | | 0.7 | | | |
| | D15 | A | 29 | 2.0 ± | 2.0 | 1 | 3 |
| | | | | 0.7 | | | |
| | | B | 29 | 1.0 ± | 1.0 | 0 | 3 |
| | | | | 0.8 | | | |
| Erythema | BL | A | 29 | 1.8 ± | 2.0 | 1 | 3 |
| | | | | 0.7 | | | |
| | | B | 29 | 1.7 ± | 2.0 | 1 | 3 |
| | | | | 0.6 | | | |
| | D15 | A | 29 | 1.4 ± | 1.0 | 0 | 3 |
| | | | | 0.7 | | | |
| | | B | 29 | 1.0 ± | 1.0 | 0 | 2 |
| | | | | 0.6 | | | |
| Edema and | BL | A | 29 | 1.0 ± | 1.0 | 0 | 2 |
| Papules | | | | 0.8 | | | |
| | | B | 29 | 1.1 ± | 1.0 | 0 | 3 |
| | | | | 0.9 | | | |
| | D15 | A | 29 | 0.6 ± | 0.0 | 0 | 2 |
| | | | | 0.8 | | | |
| | | B | 29 | 0.4 ± | 0.0 | 0 | 3 |
| | | | | 0.8 | | | |
| Weeping and | BL | A | 29 | 0.8 ± | 0.0 | 0 | 3 |
| Crusts | | | | 1.0 | | | |
| | | B | 29 | 0.9 ± | 0.0 | 0 | 3 |
| | | | | 1.0 | | | |
| | D15 | A | 29 | 0.4 ± | 0.0 | 0 | 2 |
| | | | | 0.6 | | | |
| | | B | 29 | 0.3 ± | 0.0 | 0 | 2 |
| | | | | 0.6 | | | |
| Excoriation | BL | A | 29 | 0.4 ± | 0.0 | 0 | 2 |
| | | | | 0.7 | | | |
| | | B | 29 | 0.3 ± | 0.0 | 0 | 2 |
| | | | | 0.7 | | | |
| | D15 | A | 29 | 0.4 ± | 0.0 | 0 | 2 |
| | | | | 0.7 | | | |
| | | B | 29 | 0.2 ± | 0.0 | 0 | 2 |
| | | | | 0.5 | | | |
| Lichenification | BL | A | 29 | 1.9 ± | 2.0 | 0 | 3 |
| | | | | 0.8 | | | |
| | | B | 29 | 2.0 ± | 2.0 | 0 | 3 |
| | | | | 0.7 | | | |
| | D15 | A | 29 | 1.7 ± | 2.0 | 1 | 3 |
| | | | | 0.8 | | | |
| | | B | 29 | 1.0 ± | 1.0 | 0 | 2 |
| | | | | 0.7 | | | |

Dryness will be evaluated on the non-lesional areas.
Score: 0-Absent, 1-Slight, 2-Moderate, 3-Strong

TABLE A2
Descriptive Statistics for Sum Scores, Local SCORAD

| | | | Mean ± | | | | Lower | Upper |
|---|---|---|---|---|---|---|---|---|
| Time | Code | n | SD | Median | Min | Max | CL | CL |
| BL | A | 29 | 8.2 ± | 8.0 | 6 | 15 | 7.3 | 9.1 |
| | | | 2.4 | | | | | |
| | B | 29 | 8.3 ± | 8.0 | 5 | 15 | 7.4 | 9.2 |
| | | | 2.4 | | | | | |
| D15 | A | 29 | 6.4 ± | 6.0 | 2 | 13 | 5.4 | 7.4 |
| | | | 2.6 | | | | | |
| | B | 29 | 4.0 ± | 4.0 | 0 | 13 | 2.9 | 5.0 |
| | | | 2.8 | | | | | |

TABLE A3
Descriptive Statistics for Differences to Baseline
(day 1), LOCAL SCORAD, to day 15

| | | Mean ± | | | | Lower | Upper |
|---|---|---|---|---|---|---|---|
| Code | n | SD | Median | Min | Max | CL | CL |
| A | 29 | −1.8 ± | −2.0 | −7 | 3 | −2.7 | −0.9 |
| | | 23 | | | | | |
| B | 29 | −4.3 ± | −4.0 | −11 | 2 | −5.6 | −3.1 |
| | | 3.2 | | | | | |

TABLE A4
Local SCORAD-Mean Values, Standard Deviations, and Treatment
Comparison on
Differences to BL with Paired t-Test

| | | | Mean Values ± | | p-Values |
|---|---|---|---|---|---|
| | | | SD | | Treat. Comp. |
| Time | Code | n | Sum Scores | Diff. to BL | vs. A |
| BL | A | 29 | 8.2 ± | — | — |
| | | | 2.4 | | |
| | B | 29 | 8.3 ± | — | — |
| | | | 2.4 | | |
| D15 | A | 29 | 6.4 ± | −1.8 ± | — |
| | | | 2.6 | 2.3 | |
| | B | 29 | 4.0 ± | −4.3 ± | <0.001 |
| | | | 2.8 | 3.2 | |

bold p-Value: significant (p ≤ 0.05)

What is claimed is:

1. (canceled)

2. A method of treating moderate atopic dermatitis with a local SCORAD score of at least 6, wherein the method comprises topically applying the composition on the skin at least 2 times a day, wherein the composition has a pH in the range of 2.7 to 5.0 and comprises
a polyhydroxy acid and a zinc salt of its conjugate base, wherein the polyhydroxy acid is selected from lactobionic acid, gluconolactone, or a combination of lactobionic acid and gluconolactone,
a partition coefficient enhancer selected from propylene glycol, butylene glycol, pentylene glycol, hexylene glycol, 1,5 pentane diol or a combination thereof,
a diffusion coefficient enhancer which is a C14 straight chain primary alcohol,
and wherein the composition is free of steroids.

3. (canceled)

4. A method of treating atopic dermatitis, wherein the method comprises topically applying the composition on the skin for between 10 and up to 28 days at least 2 times a day, wherein the composition has a pH in the range of 2.7 to 5.0 and comprises
a polyhydroxy acid and a zinc salt of its conjugate base, wherein the polyhydroxy acid is selected from lactobionic acid, gluconolactone, or a combination of lactobionic acid and gluconolactone,
a partition coefficient enhancer selected from propylene glycol, butylene glycol, pentylene glycol, hexylene glycol, 1,5 pentane diol or a combination thereof,
a diffusion coefficient enhancer which is a C14 straight chain primary alcohol,
and wherein the composition is free of steroids.

5. The method of claim 4, wherein the atopic dermatitis is moderate or mild atopic dermatitis.

6. The method according to claim 2, wherein the composition is applied twice daily.

7. The method according to claim 2, wherein the composition is free of topical calcineurin inhibitors.

8. The method according to claim 2, wherein the method of treating atopic dermatitis does not include treatment with a topical calcineurin inhibitor or other pharmaceutical active.

9. The method according to claim 2, wherein the composition is topically applied for 12 days and 20 days, for example, about 14 days.

10. The method according to claim 2, wherein the local SCORAD score of the atopic dermatitis at the start of treatment is at least 6, preferably between 6 and 12, for example, between 6 and 10.

11. The method according to claim 2, wherein the method involves identifying a patient suffering from moderate atopic dermatitis by measuring the local SCORAD score, determining that the local SCORAD score is at least 6, more preferably between 6 and 12.

12. The method according to claim 11, wherein the method further comprises measuring the local SCORAD score after a time period to ensure whether further treatment is needed.

13. The method according to claim 12, wherein the time period is less than 4 weeks, or less than or equal to 3 weeks, or less than or equal to 2 weeks.

14. The method according to claim 2, wherein the method of treating results in a reduction of local SCORAD score (ΔSCORAD) of at least 1, or at least 2, or at least 3, or at least 4 compared to baseline local SCORAD at the start of treatment.

15. The method according to claim 14, wherein the reduction in local SCORAD score is obtained in less than 4 weeks, or less than or equal to 3 weeks, or less than or equal to 2 weeks.

16. The method according to claim 2, wherein the pH of the composition is between 3.0 to 4.0, more preferably from 3.0 to 3.5, and even more preferably about 3.2.

17. The method according to claim 2, wherein the partition coefficient enhancer is butylene glycol.

18. The method according to claim 2, wherein the partition coefficient enhancer is present in an amount that is 15-30% w/w of the composition, or from 15-25% w/w of the composition, or from 15-20% w/w of the composition.

19. The method according to claim 2, wherein the diffusion coefficient enhancer is present in an amount that is 1.0-2.0% w/w of the composition, 1.2-1.8% w/w or about 1.5% w/w of the composition.

20. The method according to claim 2, further comprising a ceramide, preferably wherein the ceramide is present in an amount from 0.5 to 1.5% w/w of the composition.

21. The method according to claim 2, wherein the composition comprises from 3-6% w/w methyl glucose sesquistearate and 1-2% w/w PEG-20 methyl glucose sesquistearate.

22. The method according to claim 2, wherein the composition further comprises a PEG-240/HDI copolymer bis-decyltetradeceth-20 ether.

23. The method according to claim 2, wherein the composition further comprises an acrylates/C10-C30 alkyl acrylate copolymer.

24. The method according to claim 2, wherein the composition comprises
4-6% w/w polyhydroxy acid and zinc salt of its conjugate base, wherein the polyhydroxy acid is lactobionic acid
15-20% w/w partition coefficient enhancer, preferably butylene glycol
1-2% w/w diffusion coefficient enhancer, preferably tetradecyl alcohol, and
10-20% w/w emollient, preferably hydrogenated polydecene
3-12% w/w surfactant, preferably wherein the surfactant comprises one or more of methyl glucose sesquistearate, PEG-20 methyl glucose sesquistearate, glyceryl stearate or a combination thereof,
40-60% w/w water, and
up to 3% w/w humectant, preferably glycerol, and
optionally a co-surfactant, preferably in an amount up to 6% w/w of the composition
optionally a ceramide, preferably in an amount up to 1.5% w/w of the composition
optionally cholesterol, preferably in an amount up to 0.5% w/w of the composition
optionally a fatty acid, preferably in an amount up to 0.5% w/w of the composition
optionally a PEG-240/HDI copolymer bis-decyltetradeceth-20 ether, preferably in an amount up to 1% w/w of the composition
optionally a acrylates/C10-C30 alkyl acrylate copolymer, preferably in an amount up to 0.5% w/w of the composition, and
optionally a preservative, preferably in an amount up to 1% w/w of the composition wherein the pH of the composition is between 3.0 and 3.5.

25. The method according to claim 4, wherein the composition is applied twice daily.

26. The method according to claim 4, wherein the composition is free of topical calcineurin inhibitors.

27. The method according to claim 4, wherein the method of treating atopic dermatitis does not include treatment with a topical calcineurin inhibitor or other pharmaceutical active.

28. The method according to claim 4, wherein the composition is topically applied for 12 days and 20 days, for example, about 14 days.

29. The method according to claim 4, wherein the local SCORAD score of the atopic dermatitis at the start of treatment is at least 6, preferably between 6 and 12, for example, between 6 and 10.

30. The method according to claim 4, wherein the method involves identifying a patient suffering from moderate atopic dermatitis by measuring the local SCORAD score, determining that the local SCORAD score is at least 6, more preferably between 6 and 12.

31. The method according to claim 4, wherein the method of treating results in a reduction of local SCORAD score (ΔSCORAD) of at least 1, or at least 2, or at least 3, or at least 4 compared to baseline local SCORAD at the start of treatment.

32. The method according to claim 4, wherein the pH of the composition is between 3.0 to 4.0, more preferably from 3.0 to 3.5, and even more preferably about 3.2.

33. The method according to claim 4, wherein the partition coefficient enhancer is butylene glycol.

34. The method according to claim 4, wherein the partition coefficient enhancer is present in an amount that is 15-30% w/w of the composition, or from 15-25% w/w of the composition, or from 15-20% w/w of the composition.

35. The method according to claim 4, wherein the diffusion coefficient enhancer is present in an amount that is 1.0-2.0% w/w of the composition, 1.2-1.8% w/w or about 1.5% w/w of the composition.

36. The method according to claim 4, further comprising a ceramide, preferably wherein the ceramide is present in an amount from 0.5 to 1.5% w/w of the composition.

37. The method according to claim 4, wherein the composition comprises from 3-6% w/w methyl glucose sesquistearate and 1-2% w/w PEG-20 methyl glucose sesquistearate.

38. The method according to claim 4, wherein the composition further comprises a PEG-240/HDI copolymer bis-decyltetradeceth-20 ether.

39. The method according to claim 4, wherein the composition further comprises an acrylates/C10-C30 alkyl acrylate copolymer.

40. The method according to claim 4, wherein the composition comprises 4-6% w/w polyhydroxy acid and zinc salt of its conjugate base, wherein the polyhydroxy acid is lactobionic acid
15-20% w/w partition coefficient enhancer, preferably butylene glycol
1-2% w/w diffusion coefficient enhancer, preferably tetradecyl alcohol, and
10-20% w/w emollient, preferably hydrogenated polydecene
3-12% w/w surfactant, preferably wherein the surfactant comprises one or more of methyl glucose sesquistearate, PEG-20 methyl glucose sesquistearate, glyceryl stearate or a combination thereof,
40-60% w/w water, and
up to 3% w/w humectant, preferably glycerol, and
optionally a co-surfactant, preferably in an amount up to 6% w/w of the composition
optionally a ceramide, preferably in an amount up to 1.5% w/w of the composition
optionally cholesterol, preferably in an amount up to 0.5% w/w of the composition
optionally a fatty acid, preferably in an amount up to 0.5% w/w of the composition
optionally a PEG-240/HDI copolymer bis-decyltetradeceth-20 ether, preferably in an amount up to 1% w/w of the composition
optionally a acrylates/C10-C30 alkyl acrylate copolymer, preferably in an amount up to 0.5% w/w of the composition, and
optionally a preservative, preferably in an amount up to 1% w/w of the composition wherein the pH of the composition is between 3.0 and 3.5.
